(19) United States
(12) Patent Application Publication
(10) Pub. No.: US 2017/0087255 A1
(43) Pub. Date: Mar. 30, 2017

(54) IN-VIVO INTRAVASCULAR BLOOD REPLACING LIQUID, IN-VIVO INTRAVASCULAR BLOOD REPLACING LIQUID FORMULATION, AND PREFILLED SYRINGE

(71) Applicant: Terumo Kabushiki Kaisha, Tokyo (JP)

(72) Inventors: Kaori Funatsu, Saitama-shi (JP); Koji Nakamura, Hadano-shi (JP); Yoshihiko Abe, Odawara-shi (JP)

(73) Assignee: Terumo Kabushiki Kaisha, Tokyo (JP)

(21) Appl. No.: 15/281,220

(22) Filed: Sep. 30, 2016

(30) Foreign Application Priority Data
Sep. 30, 2015 (JP) 2015-195024

(51) Int. Cl. A61K 49/00; A61B 5/00; A61M 5/145; A61M 5/178; A61M 5/00

(52) U.S. Cl. CPC A61K 49/0004; A61M 5/178; A61M 5/007; A61M 5/1452; A61B 5/0066

(57) ABSTRACT

The present invention provides an in-vivo intravascular blood replacing liquid which eliminates the need for the use of a contrast agent, is subjected to a low injection resistance, and has blood immunity to a sufficient degree and continuity to some extent. An in-vivo intravascular blood replacing liquid of the present invention is injected into a blood vessel to replace blood at an in-vivo intravascular portion to be inspected therewith in making an in-vivo intravascular inspection. The blood replacing liquid contains cationic and anionic compounds, added to an aqueous medium unharmful for the living body, which are unharmful for the living body. The blood replacing liquid also contains an ion complex formed of the cationic and anionic compounds.

### BACKGROUND OF THE INVENTION

Field of the Invention

The present invention relates to an in-vivo intravascular blood replacing liquid to be used when the image of a vascular state is diagnosed, an in-vivo intravascular blood replacing liquid formulation, and a prefilled syringe.

Description of the Related Art

An intravascular diagnostic apparatus utilizing an optical technology has greatly progressed in recent years. As a representative example of apparatuses utilizing the optical technology, an optical coherence tomography (OCT) diagnostic apparatus is exemplified. By using this apparatus, it has become possible to observe the property and state (for example, cross section of blood vessel and its inner surface) of a blood vessel and in addition, visualize an image to be observed in three dimensions, having quantify the property and state of the blood vessel.

The optical coherence tomography diagnostic apparatus depicts the image of the inner surface of the blood vessel, based on light reflected from in-vivo tissues by inserting an optical fiber having a probe incorporating an optical lens and an optical mirror mounted at a front end thereof into the blood vessel and by emitting light into the blood vessel with an optical mirror disposed at the front end of the optical fiber radially scanning the inner surface of the blood vessel. In another known image diagnostic apparatus, an optical frequency domain imaging (OFDI) method called a next-generation OCT is used, as disclosed in U.S. Patent Application Publication 2011245683 (A1) (patent document 1 proposed by the present applicant).

When the intravascular image diagnosis is conducted, a catheter for use in the intravascular image diagnosis is delivered to a portion to be observed through a guide wire. In performing the intravascular image diagnosis, reflection of light and ultrasonic waves may occur owing to blood containing blood cell components such as red blood cells, which hinders the formation of tomographic and inner surface images of a blood vessel to be diagnosed with high accuracy. Thus in performing the intravascular image diagnosis, it is necessary to remove the blood cell components from the blood vessel to be diagnosed.

At a clinical site, image diagnosis is carried out after a state in which the blood cell components are removed from the blood vessel is temporarily produced by injecting a liquid such as a contrast agent having a high viscosity into the blood vessel. An operation of discharging the liquid to the blood vessel is called a flush operation. The liquid discharged in the flush operation is called a flush solution.

The present applicant proposed the flush solution (in-vivo intravascular blood replacing liquid) as disclosed in Japanese Patent Application Laid-Open Publication No. 2015-10065 (patent document 2). The in-vivo intravascular blood replacing liquid 1 of the patent document 2 is injected into a blood vessel to replace blood of an in-vivo intravascular portion to be inspected with the in-vivo intravascular blood replacing liquid in conducting intravascular inspection.

The blood replacing liquid 1 is a transparent aqueous liquid consisting of an aqueous medium unharmful for a living body and hydrophilic macromolecules added to the aqueous medium to enhance the viscosity thereof. As the hydrophilic macromolecules to be used, polyethylene glycol, ficoll, polyvinyl alcohol, styrene-maleic anhydride alternating copolymers, divinyl ether-maleic anhydride alternating copolymers, polyvinylpyrrolidone, polyvinyl methyl ether, polyvinyl methyl oxazoline, poly ethyl oxazoline, poly hydroxypropyl oxazoline, poly hydroxypropyl methacrylamide, polymethacrylamide, polydimethylacrylamide, poly hydroxypropyl methacrylate, polyhydroxyethyl acrylate, hydroxymethyl cellulose, hydroxyethyl cellulose, polyaspartamide, and synthetic polyamino acid are exemplified.

The present applicant proposed the gel composition and its use as disclosed in WO2012/102210 (patent document 3). In the patent document 3, the present applicant proposed the gel composition which can be easily formed matrix (gel) in a non-organic solvent and allows a medicine to be easily enclosed in a syringe and the sustained release formulation to be prepared at a necessary time and the use thereof.

More specifically, there is disclosed in the patent document 3 the gel composition containing the complex of the glycyrrhizin acid and the cationic substance (for example, thiamine). In the disclosure made in the patent document 3, the gel composition comprises the component applied to a living body and is particularly useful as a carrier of a gel for use in a pharmaceutical formulation, namely, useful as a matrix for enclosing a medicine therein and controlling discharge of the enclosed medicine.

The use of the contrast agent as the flush solution may cause a side effect represented by contrast nephropathy. Thus a decrease of the amount of the contrast agent is demanded. Because the flush solution is injected into the blood vessel through a guiding catheter, the viscosity of the flush solution greatly affects an injection resistance force. The resistance to the injection of the flush solution is greatly affected by the inner diameter of the catheter serving as the flow path of the flush solution. When the resistance to the injection of the flush solution is excessively high, there is a possibility that the flush solution is defectively injected into the blood vessel.

The in-vivo intravascular blood replacing liquid disclosed in the patent document 2 is safe for a living body in that a contrast agent is not used. But it is desirable that the blood replacing liquid has a lower viscosity so that it is subjected to a low injection resistance. The gel composition containing the complex of the glycyrrhizin acid and the cationic substance (for example, thiamine) is disclosed in the patent document 3. But the use of the gel composition as the in-vivo intravascular blood replacing liquid is not disclosed or suggested.

### SUMMARY OF THE INVENTION

Therefore it is an object of the present invention to provide an in-vivo intravascular blood replacing liquid which eliminates the need for the use of a contrast agent, is subjected to a low injection resistance, and has blood removability to a sufficient degree and continuity to some extent, an in-vivo intravascular blood replacing liquid formulation, and a prefilled syringe.

The means for achieving the above-described object of the present invention is as described below.

An in-vivo intravascular blood replacing liquid injected into a blood vessel to replace blood at an in-vivo intravascular portion to be inspected therewith in making an in-vivo intravascular inspection, wherein said blood replacing liquid comprises an aqueous medium unharmful for a living body and a cationic compound and an anionic compound, added thereto, which are unharmful for said living body; and said blood replacing liquid includes an ion complex formed of said cationic compound and said anionic compound.

### BRIEF DESCRIPTION OF THE DRAWINGS

FIG. 1 is an explanatory view for explaining a state in which an in-vivo intravascular blood replacing liquid of the present invention is administered to a blood vessel.

FIG. 2 is a front view of a prefilled syringe of the present invention.

FIG. 3 is a vertical sectional view of the prefilled syringe shown in FIG. 2.

FIG. 4 is a front view of one example of a guiding catheter to which the prefilled syringe of the present invention can be connected.

FIG. 5 is a front view of one example of an in-vivo insertion probe for an intravascular optical coherence tomography diagnostic apparatus for which the in-vivo intravascular blood replacing liquid of the present invention is used.

FIG. 6 is an enlarged vertical sectional view of a front end portion of the in-vivo insertion probe for the intravascular optical coherence tomography diagnostic apparatus shown in FIG. 5.

FIG. 7 is a front view of one example of an in-vivo insertion probe for an intravascular ultrasonic diagnostic apparatus for which the in-vivo intravascular blood replacing liquid of the present invention is used.

FIG. 8 is an enlarged vertical sectional view of a front end portion of the in-vivo insertion probe for the intravascular ultrasonic diagnostic apparatus shown in FIG. 7.

FIG. 9 shows a ¹H-NMR spectrum of an in-vivo intravascular blood replacing liquid of one embodiment of the present invention in the vicinity of 7.9 ppm.

FIG. 10 shows a ¹H-NMR spectrum of an in-vivo intravascular blood replacing liquid of another embodiment of the present invention in the vicinity of 7.9 ppm.

FIG. 11 shows a ¹H-NMR spectrum of an in-vivo intravascular blood replacing liquid of still another embodiment of the present invention in the vicinity of 7.9 ppm.

FIG. 12 shows a ¹H-NMR spectrum of an in-vivo intravascular blood replacing liquid of a different embodiment of the present invention in the vicinity of 7.9 ppm.

### DESCRIPTION OF THE PREFERRED EMBODIMENT

An in-vivo intravascular blood replacing liquid of the present invention, an in-vivo intravascular blood replacing liquid formulation using it, and a prefilled syringe are described below with reference to embodiments.

In making an in-vivo intravascular inspection, an in-vivo intravascular blood replacing liquid of the present invention is injected into a blood vessel to replace blood at an in-vivo intravascular portion to be inspected. The blood replacing liquid 1 of the present invention comprises an aqueous medium unharmful for a living body and a cationic compound and an anionic compound, added thereto, which are unharmful for the living body. The in-vivo intravascular blood replacing liquid of the present invention contains an ion complex formed of the cationic compound and the anionic compound.

The in-vivo intravascular blood replacing liquid (so-called flush solution) of the present invention contains a proper amount of an ion complex formed of the cationic compound and the anionic compound. Therefore the in-vivo intravascular blood replacing liquid of the present invention is low in its viscosity and has a low injection resistance and thus can be easily injected into the blood vessel. After the blood replacing liquid is injected into the blood vessel, it displays a sufficient blood pressing performance owing to the presence of the ion complex contained therein and stays for a certain period of time in a portion of the blood vessel to which the blood replacing liquid has been injected. Thus, by using an in-vivo insertion probe for image diagnosis, it is possible to obtain the information for blood vessel image diagnosis without being adversely affected by blood.

The in-vivo intravascular blood replacing liquid formulation of the present invention comprises a medical container and the above-described intravascular blood replacing liquid filled therein.

The intravascular blood replacing liquid filled inside the medical container has the above-described effect. It is very easy to perform a preparation operation of administering the blood replacing liquid of the in-vivo intravascular blood replacing liquid formulation into the living body.

A prefilled syringe of the present invention includes an outer cylinder, a gasket slidably accommodated inside the outer cylinder, a sealing part for sealing a front end portion of the outer cylinder, and the intravascular blood replacing liquid filled inside the outer cylinder.

The intravascular blood replacing liquid filled inside medical container has the above-described effect. The prefilled syringe allows the intravascular blood replacing liquid to be administered very easily into the living body.

A prefilled syringe 10 in which the intravascular blood replacing liquid of the present invention has been filled comprises an outer cylinder 12, a gasket 16 slidably accommodated inside the outer cylinder 12, a sealing part 15 for sealing a front end portion of the outer cylinder 12, and the intravascular blood replacing liquid 1 filled inside the outer cylinder.

It is preferable to use the outer cylinder, the gasket, and the sealing part all subjected to sterilization in advance. The sterilizing method is not specifically limited. For example, it is possible to use a high-pressure steam sterilization method, a dry heating sterilization method, an ethylene oxide gas sterilization method, a radiation (for example, electron beam, x-ray, γ-ray, and the like) sterilization method, a sterilization method to be carried out by using ozone water, and a sterilization method to be carried out by using a hydrogen peroxide solution.

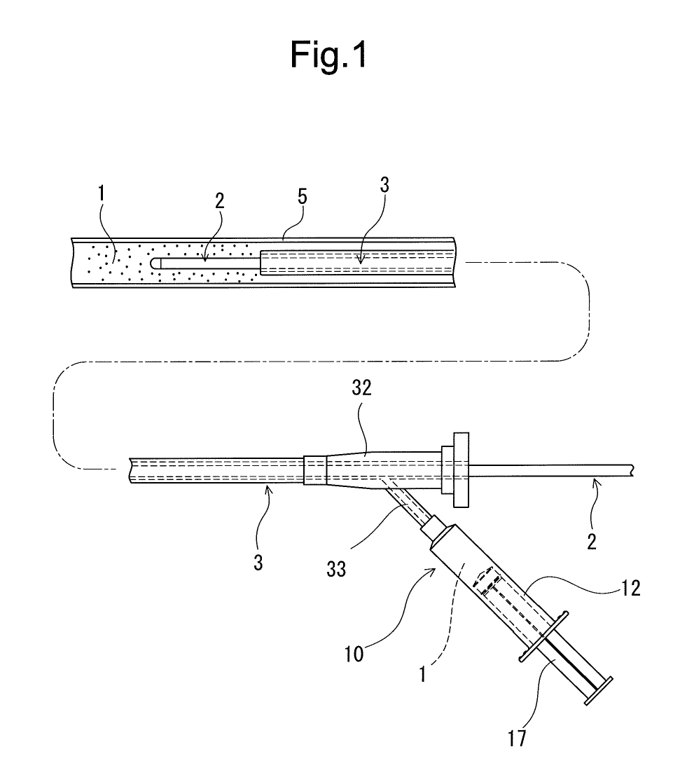

As shown in FIG. 1, the in-vivo intravascular blood replacing liquid 1 and prefilled syringe 10 of the present invention are used by injecting the in-vivo intravascular blood replacing liquid into the blood vessel by using a tubular body (for example, catheter, probe) inserted into the in-vivo intravascular portion.

With reference to FIG. 1, a guiding catheter 3 is inserted into a blood vessel 5. An in-vivo insertion probe 2 for image diagnosis is inserted into the guiding catheter. The prefilled syringe 10 in which the intravascular blood replacing liquid has been filled is mounted on a side port 33 of a hub 32 of the guiding catheter 3.

After a front end portion of the in-vivo insertion probe 2 for image diagnosis is disposed in the vicinity of the portion to be diagnosed, a plunger 17 of the prefilled syringe 10 mounted on the guiding catheter 3 is pressed into the prefilled syringe. Thereby the intravascular blood replacing liquid 1 passes inside the guiding catheter 3 and is injected into the blood vessel from the front end of the guiding catheter. Blood at the portion to be diagnosed is carried away by the intravascular blood replacing liquid 1 injected into the blood vessel. As a result, the portion where the intravascular blood replacing liquid has been injected is filled with the intravascular blood replacing liquid. Thereby it is possible to obtain information for blood vessel diagnosis by the in-vivo insertion probe 2 for image diagnosis without being adversely affected by blood.

The in-vivo intravascular blood replacing liquid 1 of the present invention comprises the aqueous medium unharmful for the living body. The cationic compounds and the anionic compound are added to the aqueous medium. The blood replacing liquid includes an ion complex formed of the cationic compound and the anionic compound. The ion complex is formed in the aqueous medium.

As the aqueous medium, sterile water, saline, and a buffer solution are preferably used. As the sterile water, water for injection, distilled water and RO water are preferable. The viscosity of the in-vivo intravascular blood replacing liquid 1 is favorably not more than 2 mPa·s at not more than 30 degrees C. It is also preferable that the in-vivo intravascular blood replacing liquid 1 displays the gelling property at not less than 25 degrees C.

It has been regarded that a flush solution having a viscosity not less than 2 mPa·s is effective for flushing blood. But as a result of investigations, it has been confirmed that even a liquid having a viscosity less than 2 mPa·s is capable of removing the blood, provided that the flush solution contains the ion complex.

It is preferable that the viscosity of the in-vivo intravascular blood replacing liquid is not more than 2 mPa·s at least 30 degrees C. In the case of the in-vivo intravascular blood replacing liquid having the viscosity in the above-described range, an injection resistance is low in injecting it into the blood vessel and thus it can be easily injected thereinto.

As the anionic compound to be used in the present invention, it is preferable to use at least one selected from the group consisting of glycyrrhizin acids, hyaluronic acids, chondroitin sulfates, alginic acids, ammonium sulfates, dextran sulfates, and glucuronic acids. As the acids of the present invention, not only the above-described acids, but also derivatives thereof and salts thereof are included.

As the anionic compound, glycyrrhizin acids are especially preferable. The glycyrrhizin acids include glycyrrhizin acid and its derivatives or salts thereof. As the glycyrrhizin acids, it is possible to exemplify glycyrrhizin acid, methyl glycyrrhizinate, stearyl glycyrrhizinate, nitric acid ester of glycyrrhizin acid, acetic acid ester of glycyrrhizin acid, alkali metal salts such as disodium glycyrrhizinate, trisodium glycyrrhizinate, dipotassium glycyrrhizinate, and tripotassium glycyrrhizinate, and ammonium salts such as monoammonium glycyrrhizinate. These glycyrrhizin acids can be used singly or in combination of two or more kinds thereof. As the salts, it is possible to exemplify organic acid salts such as acetates, trifluoroacetates, fumarates, maleates, tartrates, citrates, methanesulfonates, toluene sulfonates, lactates, gluconates, aspartates, and oxalates; inorganic acid salts such as hydrochlorides, hydrobromide, sulfates, and phosphates; salts formed as a result of reaction between acids and organic salts such as trimethylamine salts, triethylamine salts, monoethanolamine salts, triethanolamine salts, and salts such as pyridine salts formed as a result of reaction between acids and tertiary amine; salts formed as a result of reaction between acids and inorganic salts such as ammonium salts, alkali metal salts such as sodium salts, potassium salts, and the like, and alkaline-earth metal salts such as calcium salts, magnesium salts, and the like, and aluminum salts.

The cationic compound to be used in the present invention includes basic amino acids or derivatives thereof, chitin or chitosan or derivatives thereof, and basic compounds. Thiamines which are basic compounds are especially preferable. The thiamines include thiamine and derivatives thereof or the salts thereof. As the thiamines, thiamine chloride hydrochloride is especially preferable. As the thiamines, thiamine, thiamine disulfide, fursultiamine, bisthiamine, dicethiamine, bisbentiamine, bisibuthiamine, benfotiamine, and salts of these thiamines are listed. As the salts of the thiamines, nitrates, hydrochlorides, sulfates, and phosphates are listed. The thiamine has a peak in the vicinity of 7.9 ppm in the ¹H-NMR spectrum. The half width of the thiamine in the ¹H-NMR spectrum is 2.1 (7.86 to 7.88 ppm) Hz.

As combinations of the cationic and anionic compounds, mixtures of the glycyrrhizin acids and the basic amino acids or the derivatives thereof, mixtures of the chitin or the chitosan or derivatives thereof and the hyaluronic acid or derivatives thereof, and mixtures of chondroitin sulfate or derivatives thereof and the basic compounds are preferable. It is especially preferable to combine the glycyrrhizin acids and the thiamine with each other.

The blood replacing liquid 1 of the present invention contains the ion complex formed of the cationic and anionic compounds. The half width of the peak derived from any one of compounds (more specifically, peak derived from cationic compound or anionic compound in the blood replacing liquid 1) in the ¹H-NMR spectrum is favorably 3 to 15 Hz and especially favorably 3 to 10 Hz. The half width of the peak derived from any one of compounds (more specifically, peak derived from cationic compound or anionic compound in the blood replacing liquid 1) in the ¹H-NMR spectrum of the blood replacing liquid is favorably 1.5 to 20 times and more favorably 1.5 to 5 times as large as the half width of the peak derived from the cationic compound alone or the anionic compound alone in the ¹H-NMR spectrum (in other words, the cationic compound or the anionic compound in the ¹H-NMR spectrum when the blood replacing liquid contains only the cationic compound or the anionic compound).

In the blood replacing liquid 1, the mol ratio between the cationic compound and the anionic compound is set to preferably 1:4 to 4:1 and especially favorably 2:1 to 1:2. The property of the blood replacing liquid containing the cationic and anionic compounds varies according to not only the concentration of the cationic compound and that of the anionic compound, but also the mol ratio therebetween. At a specific mol ratio therebetween, the cationic compound and anionic compounds firmly combine with each other. As a result, the formation rate of the ion complex becomes high and the viscosity thereof becomes high. In this case, the ion complex may be inappropriate for the blood replacing liquid because the ion complex has a high injection resistance.

As a result of intensive investigations made by the present inventors, although the gel state of the ion complex is discussed in terms of viscosity, they have found that it is possible to grasp the gel state in terms of a specific half width of a peak in an NMR spectrum of a compound. Hydrogen (proton) contained in a functional group involved in a hydrogen bond repeats desorption and bonding. Thus it is difficult to observe the peak of the protons. But it is possible to observe the peak of other protons present in the compound. The other protons are not directly involved in the hydrogen bonding. But as the bonding force between molecules becomes stronger, the peak of the other protons tends to become increasingly broad. As a reason, it is assumed that as an ionic bond between molecules becomes stronger, the distance between the molecules becomes increasingly short, which causes protons not involved in the ionic bond between molecules to interact with atoms of molecules disposed on the periphery thereof. Therefore the present inventors have found that the bonding force between the cationic and anionic compounds contained in the blood replacing liquid can be defined by the half width of a chemical shift not involved in the bonding between the cationic and anionic compounds in NMR measurement.

The relationship between ppm which indicates the peak and the unit Hz of the half width is as follows: The ppm is called a chemical shift and indicates the appearance position of the peak observed in the NMR spectrum. An NMR is an apparatus which generates a strong magnetic field (normally, 500 MHz). When a sample is placed in the magnetic field, an electronic state in the sample temporarily goes into a high energy state. Each atom of the compound has a specific resonance frequency. The amount of a shift of the specific resonance frequency from that of a reference is indicated by the chemical shift (ppm). The half width is the width of the peak in the chemical shift. Each constituent atom of the compound has its own specific resonance frequency. In principle, the peak is supposed to be sharp. But each atom is electronically affected by other atoms disposed close thereto according to the difference in a situation where molecules are placed. Conceivably, owing to this influence, the peak becomes broad and thus the half width becomes broad.

The ion complex formed of the cationic and anionic compounds is described below by using the cationic compound having amino groups and the anionic compound having carboxylic groups. The amino group of the cationic compound and the carboxylic group of the anionic compound undergo hydrogen bonding respectively to form the ion complex. The blood replacing liquid containing the ion complex displays an effective blood removal function although it has a low viscosity. Normally the blood replacing liquid removes blood by utilizing its high viscosity.

In the blood replacing liquids containing the ion complex, the viscosities and blood removing functions thereof vary according to a combination of the cationic compound and the anionic compound and a mol ratio therebetween. A compound containing the cationic and anionic compounds whose molecular size is large and having a large number of substituent groups of hydroxyl groups and the amino groups has a high viscosity. This is because the ion complex goes into a gel state. An index for evaluating the gel state is for example a viscosity. It is assumed that when an aqueous liquid containing the cationic and anionic compounds has a viscosity of not less than 3.0 mPa·s, the ion complex formed in the liquid has gone into the gel state.

As a result of the present inventors' intensive investigations, it has been found that the blood replacing liquid of the present invention, consisting of an aqueous liquid, which has favorable properties has a half width (specifically, not less than 3 Hz) of a specific peak (for example, peak derived from cationic compound) in the NMR spectrum of the compound contained therein. It has been also found that the formation state of the ion complex, in other words, the formation extent of the gel is effective as the blood replacing liquid having the half width in the above-described range.

Because the carboxylic group and the amine group involved in the hydrogen bonding have a strong binding force, the hydrogen (proton) thereof cannot be observed by the NMR. But the NMR is capable of observing the peak of other protons present in the compound. The other protons are not directly involved in the hydrogen bonding. But as the bonding force between molecules becomes stronger, the peak of the protons tends to become increasingly broad. As a reason, it is assumed that as the bonding force between the molecules, namely, the ionic bond between the molecules becomes stronger, the distance between the molecules becomes increasingly short, which causes protons in molecules not directly involved in the ionic bond to interact with atoms of other molecules disposed on the periphery thereof.

The present inventors have focused attention to a cationic compound which allows a peak to be easily detected and the half width to be easily measured and found a preferable half width. An example of the ion complex is described below by using the thiamines and the glycyrrhizin acids. In an aqueous solution containing the thiamines and the glycyrrhizin acids at an equimolar ratio, the half width derived from the thiamines at 7.9 ppm is twice as large as that derived from the thiamines singly contained in an aqueous solution. It has been confirmed that although the viscosities of both aqueous solutions are not greatly different from each other, the ion complex solution containing the thiamines and glycyrrhizin acids at an equimolar ratio has image diagnosis performance superior to that of the aqueous solution containing only the thiamines.

As a result of the present inventors' investigations, they have confirmed the following:

The half width is not necessarily maximum in the aqueous solution containing the cationic and anionic compounds at an equimolar ratio. As the concentration of the ion complex becomes higher, intermolecular interactions are liable to increasingly occur. Thus the half width becomes large. The higher is the ratio of the molecular size of the cationic compound to that of the anionic compound or the higher is the ratio of the molecular size of the anionic compound to that of the cationic compound or the higher is the ratio of cations having a large number of functional groups to anions, the half width tends to become increasingly large. There is a correlation between an increase of the half width and the viscosity. Thus when the half width becomes too large, there is a fear that the viscosity causes the solution to have an injection resistance inappropriate for practical use. Thus it is preferable to mix cations and anions with each other at the above-described proper mixing ratio (mol ratio).

In an experiment, the glycyrrhizin acid was larger than the thiamines in the molecular size thereof and in the number of carbonyl groups. Thus the higher was mol ratio of the glycyrrhizin acid, the larger was the half width and viscosity. In a case where an ion complex solution of glycyrrhizin acid/thiamine, when the half width at 7.9 ppm in the thiamine NMR spectrum became not less than 15.0 Hz, it has been confirmed that the ion complex solution had a viscosity of not less than 3.0 mPa·s and a high injection resistance.

As a result of the present inventors' further investigations, they were found that the following matters.

The in-vivo intravascular blood replacing liquid of the present invention has a half width (full width at half maximum) of a peak derived from a cationic compound (for example, derived from thiamines) in a ¹H-NMR spectrum. In the in-vivo intravascular blood replacing liquid, the half width (full width at half maximum) of a peak derived from a cationic compound (for example, derived from thiamines) of the blood replacing liquid in the ¹H-NMR spectrum is favorably 3.0 to 15.0 Hz. In the in-vivo intravascular blood replacing liquid, the half width (full width at half maximum) of a peak derived from a cationic compound (for example, derived from thiamines) of the blood replacing liquid in the ¹H-NMR spectrum is favorably 1.5 to 20 times, more favorably 2.0 to 12 times, and especially favorably 2.0 to 5 times as large as the half width of the peak derived from the cationic compound alone (thiamines themselves).

The in-vivo intravascular blood replacing liquid 1 may contain the hydrophilic macromolecules as a viscosity modifier thereof. Although the kind of the hydrophilic macromolecules is not specifically limited, it is preferable that the hydrophilic macromolecules do not contain dextran and is water-soluble. The hydrophilic macromolecules have a structure in which monomers having the same structure are repeatedly arranged.

Examples of the hydrophilic macromolecules of the present invention include gelatin, methylcellulose, polyvinylpyrrolidone, polyethylene glycol, ficoll, polyvinyl alcohol, styrene-maleic anhydride alternating copolymers, divinyl ether-maleic anhydride alternating copolymers, polyvinyl methyl ether, polyvinyl methyl oxazoline, poly ethyl oxazoline, poly hydroxypropyl oxazoline, poly hydroxypropyl methacrylamide, polymethacrylamide, polydimethylacrylamide, poly hydroxypropyl methacrylate, polyhydroxyethyl acrylate, hydroxymethyl cellulose, hydroxyethyl cellulose, polyaspartamide, or synthetic polyamino acid.

The hydrophilic macromolecules may be dissolved in a solvent in advance before the macromolecules are mixed with the aqueous medium. Solvents which dissolve the hydrophilic macromolecules therein are not specifically limited. But considering that it is necessary for the solvent to mix with water, water, alcohols, DMF, THF, and DMSO are desirable. Of these solvents, water is most desirable. Hydrophilic macromolecules having a molecular weight of 200 to 1000000 are favorable and those having a molecular weight of 400 to 40000 are especially favorable.

The in-vivo intravascular blood replacing liquid 1 may contain glucose and sodium chloride as its additives. According to the present inventors' finding, it is assumed that the addition of the glucose and the sodium chloride to the blood replacing liquid 1 affects the state of the ion complex formed of the cationic compound and the anionic compound. More specifically, it is assumed that the addition of the glucose and the sodium chloride thereto affects the cationic compound (thiamines) weakens the state of the ion complex and that the anionic compound (glycyrrhizin acid) strengthens the state of the ion complex. The presence of these additives in the blood replacing liquid 1 allows the blood replacing liquid 1 to have an osmotic pressure and viscosity close to those of blood.

According to the present inventors' finding obtained when the glycyrrhizin acid was used as the anionic compound and the thiamines were used as the cationic compound, as the pH of the in-vivo intravascular blood replacing liquid becomes higher, the half width of the peak becomes increasingly small. Thus it has been found that to allow a favorable interaction to occur between the cationic and anionic compounds, it is necessary to set the pH of the blood replacing liquid to an acidic side, specifically 3 to 5 and especially favorably 3.5 to 4.5 and that in the case of a compound having its pKa in the same pH region, it is desirable to set the pH of the blood replacing liquid within the same pH region.

The in-vivo intravascular blood replacing liquid formulation of the present invention comprises the medical container and the above described intravascular blood replacing liquid filled therein. As the medical container, it is possible to use a container for transfusion such as a soft bag, a pouch container, a plastic bottle; a vial, an ample, and a prefilled syringe.

As the container to be used to fill the intravascular blood replacing liquid of the present invention therein, it is possible to use containers satisfying regulations demanded in each country.

As the container to be used to fill the intravascular blood replacing liquid of the present invention therein, it is possible to use the above-described medical containers generally used. In view of the use of the intravascular blood replacing liquid of the present invention, a prefilled syringe formulation which is described later is preferable.

In a case where the medical container is a bag, it is possible to preferably use the medical container formed of any of various resins such as polypropylene, polyethylene, polystyrene, polyamide, polycarbonate, polyvinyl chloride, poly-(4-methylpentene-1), acrylic resin, an acrylonitrile-butadiene-styrene copolymer, polyester including polyethylene terephthalate; and cyclic polyolefins. In a case where a bag is selected as the container for the intravascular blood replacing liquid, it is necessary to transfer the intravascular blood replacing liquid to a syringe at a medical front. In a case where the medical container is the prefilled syringe, the material to be selected for the outer cylinder of the syringe is not specifically limited. It is possible to preferably use the outer cylinder of the syringe formed of any of various resins such as polypropylene, polyethylene, polystyrene, polyamide, polycarbonate, polyvinyl chloride, poly-(4-methylpentene-1), acrylic resin, an acrylonitrile-butadiene-styrene copolymer, polyester including polyethylene terephthalate; cyclic olefin copolymers; and cyclic polyolefins.

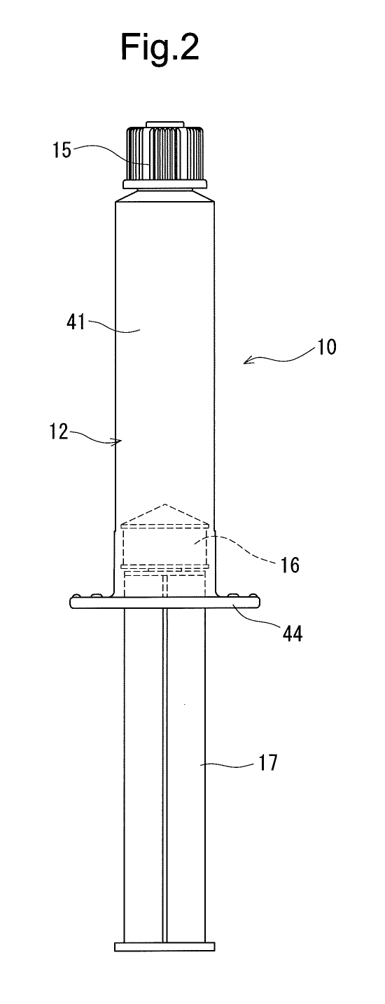

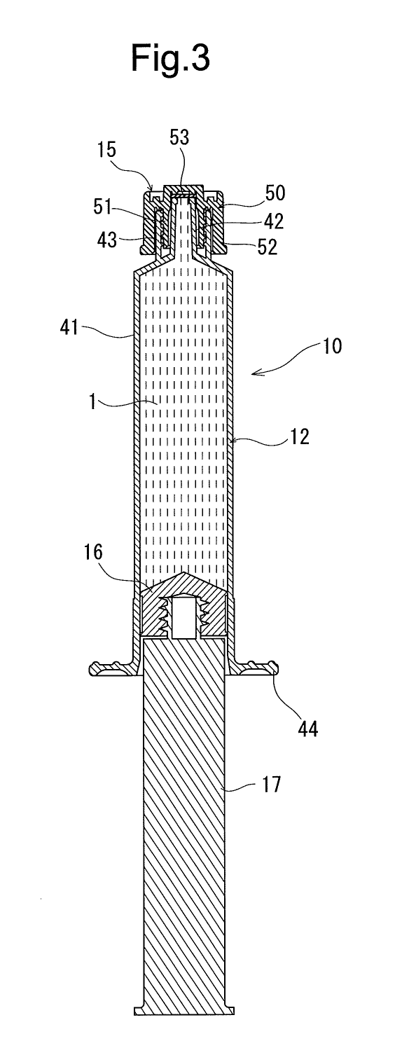

As shown in FIGS. 2 and 3, the prefilled syringe 10 of the present invention in which the intravascular blood replacing liquid has been filled comprises the outer cylinder 12, the gasket 16 slidably accommodated inside the outer cylinder 12, the sealing part 15 for sealing the front end portion of the outer cylinder 12, the intravascular blood replacing liquid 1 filled inside the outer cylinder, and the plunger 17 mounted on the gasket 16.

It is preferable to subject the prefilled syringe 10 to heat sterilization (autoclave sterilization) with the intravascular blood replacing liquid being filled therein. More specifically, as shown in FIGS. 2 and 3, it is preferable to subject the prefilled syringe 10 to the steam sterilization in a state where the intravascular blood replacing liquid is filled in the outer cylinder 12 whose front end portion thereof is sealed with the sealing member 15 and the rear end side of the outer cylinder 12 is sealed with the gasket 16 accommodated in the outer cylinder 12. The prefilled syringe 10 in which the intravascular blood replacing liquid is filled is subjected to the autoclave sterilization by exposing the prefilled syringe 10 to an atmosphere having a temperature of 118 to 122 degrees C. and 0.8 to 2.0 kg/cm² for 15 to 30 minutes.

In a case where the components of the intravascular blood replacing liquid is not heat-resistant, the intravascular blood replacing liquid is filled in a sterilized syringe under a sterilized atmosphere.

The outer cylinder 12 has an outer cylinder body part 41, a nozzle portion 42 formed at a front end portion of the outer cylinder body part 41, and a flange part 44 formed at a rear end portion of the outer cylinder body part 41.

The outer cylinder 12 is a tubular body formed of a transparent or semitransparent material. It is preferable that the material thereof has a low degree of oxygen permeability and hydrogen permeability.

The outer cylinder body part 41 is a substantially tubular part accommodating the gasket 16 liquid-tightly and slidably. The nozzle portion 42 is a tubular portion whose diameter is smaller than that of the outer cylinder body part 41. The diameter of the front end portion (shoulder portion) of the outer cylinder body part 41 decreases in a tapered configuration toward the nozzle portion 42. The outer cylinder 12 of this embodiment has a collar portion 43 surrounding the nozzle portion 42. The nozzle portion 42 is formed at the front end of the outer cylinder 12 and has an opening for discharging such as a liquid medicine filled inside the outer cylinder at its front end. The diameter of the nozzle portion decreases in a tapered configuration toward its front end. The collar portion 43 is formed cylindrically and concentrically with the nozzle portion 42 in such a way as to surround the nozzle portion 42. A spiral groove portion engageable with a spiral projected portion formed on an outer circumferential surface of a nozzle portion accommodation part 51 of the seal cap 15 which is a seal member to be described later and with a projected portion formed at a rear end of the side port 33 of the guiding catheter 3 is formed on an inner circumferential surface of the collar portion 43. The flange part 44 is an elliptic donut-shaped disk part projected vertically to the outer cylinder 12 from the entire circumference of the rear end thereof. The flange part 44 has two opposed gripping portions having a wide width.

As materials for forming the outer cylinder 12, it is possible to list various resins such as polypropylene, polyethylene, polystyrene, polyamide, polycarbonate, polyvinyl chloride, poly-(4-methylpentene-1), acrylic resin, an acrylonitrile-butadiene-styrene copolymer, polyester including polyethylene terephthalate; and cyclic olefin copolymers; and cyclic polyolefin. Of these resins, the polypropylene, the cyclic olefin copolymers, and the cyclic polyolefin are preferable because these resins are easily moldable and heat-resistant.

The seal cap 15 serving as the seal member comprises a cap body part 50 and a seal member 53 accommodated inside the cap body part. As shown in FIG. 3, the cap body part 50 is formed in the shape of a cap and has a nozzle portion accommodation part 51, a collar portion accommodation part 52, and a seal member holding part formed on an inner surface of the nozzle portion accommodation part 51. The nozzle portion accommodation part 51 is a tubular part formed at a central portion of the seal cap 15 and is closed at one end thereof and open at the other end thereof. An inner diameter of the nozzle portion accommodation part 51 is substantially equal from its one end to its other end. The spiral projected portion engageable with the spiral groove portion formed on the inner circumferential surface of the collar portion 43 of the outer cylinder 12 is formed on the outer circumferential surface of the nozzle portion accommodation part 51.

As materials for forming the seal cap, it is possible to list various resins such as polypropylene, polyethylene, polystyrene, polyamide, polycarbonate, polyvinyl chloride, poly-(4-methylpentene-1), acrylic resin, an acrylonitrile-butadiene-styrene copolymer, polyester including polyethylene terephthalate; cyclic olefin copolymers, and cyclic polyolefins. As materials for forming the seal member 53, it is preferable to use natural rubber, synthetic rubber such as isoprene rubber, butadiene rubber, fluororubber, and silicone rubber; and thermoplastic elastomers such as olefin-based elastomers and styrene-based elastomers.

As shown in FIGS. 2 and 3, the gasket 16 has a body part extended substantially equally in its outer diameter and a plurality of annular ribs (in this embodiment, two annular ribs are formed) formed on the body part of the gasket. The annular ribs liquid-tightly contact an inner surface of the outer cylinder 12. A front end surface of the gasket 16 has a configuration corresponding to that of the inner surface of the front end of the outer cylinder 12 so as to prevent a gap from being formed as much as possible between the front end surface of the gasket and the inner surface of the front end of the outer cylinder when both surfaces contact each other. The gasket 16 has a plunger mounting part on its rear end portion. In this embodiment, the plunger mounting part is constructed of a concave portion extended inward from the rear end portion of the gasket and a female screw portion formed on an inner surface of the concave portion.

As a material for forming the gasket 16, it is preferable to use elastic rubber (for example, butyl rubber, latex rubber, silicone rubber) or synthetic resin (for example, styrene elastomer such as SBS elastomer, SEBS elastomer; and polyolefin elastomer such as ethylene-α-olefin copolymer).

The plunger 17 has a projected portion tubularly projected from its front end. A male screw portion which engages the concave portion of the gasket 16 is formed on an outer surface of the projected portion. The plunger 17 has a sectionally cross-shaped body part axially extended and a pressing disk part formed at a rear end portion thereof. In this embodiment, although the plunger 17 is mounted on the gasket in advance, the form of the plunger is not limited to that. The plunger may be mounted on the gasket when the prefilled syringe is used.

Description is made below on a guiding catheter and an in-vivo insertion probe for image diagnosis for which the in-vivo intravascular blood replacing liquid of the present invention is used.

The guiding catheter and the in-vivo insertion probe for image diagnosis shown in the drawings are examples and not limited to the form described below.

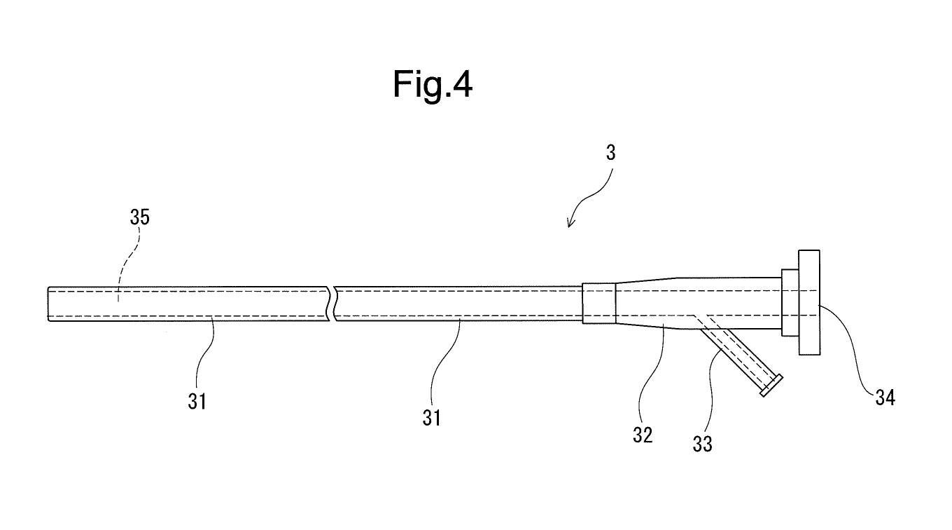

The guiding catheter 3 shown in FIG. 4 is composed of a catheter tube 31 which is hollow and has a predetermined length and a branch hub 32 mounted on a rear end portion of the catheter tube 31. The catheter 3 has a lumen 35 extended from a front end of the catheter tube 31 to an open portion 34 of the branch hub 32. The open portion 34 of the branch hub 32 is used as an insertion opening for the in-vivo insertion probe for image diagnosis. The side port 33 of the branch hub 32 is used as a connection port to which the prefilled syringe 10 in which the intravascular blood replacing liquid has been filled is connected.

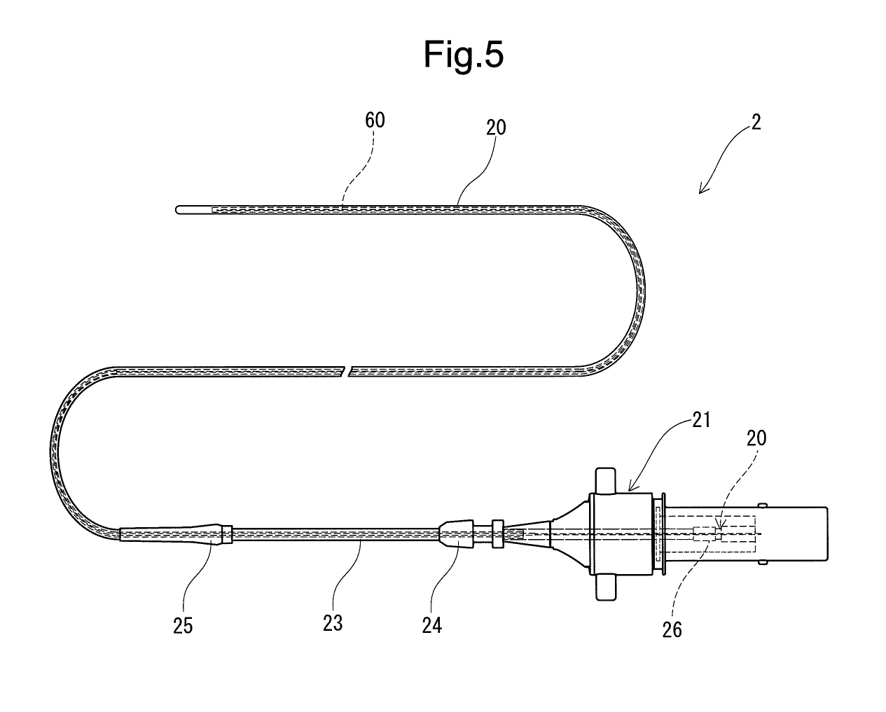

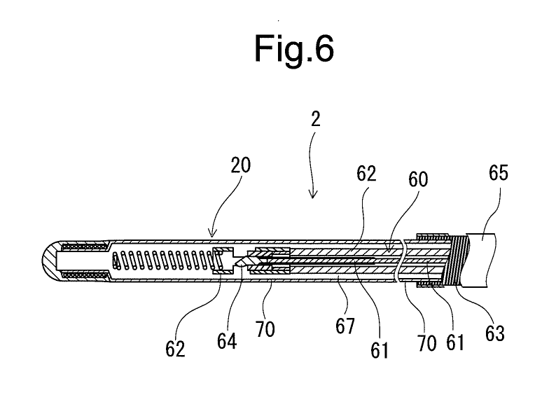

The in-vivo insertion probe for image diagnosis shown in FIGS. 5 and 6 comprises a sheath 20 to be inserted into a body cavity (inside guiding catheter) and a data acquisition shaft 60 inserted into the sheath 20. The data acquisition shaft 60 has a drive transmission hollow shaft 62 and an optical fiber 61 penetrating through the hollow shaft 62 and having a chip portion 64 exposed from a front end portion of the hollow shaft 62. The data acquisition shaft 60 is rotated by a rotational force imparted thereto at a proximal portion thereof.

The optical in-vivo insertion probe 2 of this embodiment has the data acquisition shaft 60, the sheath 20 for accommodating the data acquisition shaft, and an operation member 21 through which the data acquisition shaft penetrates and which is positioned nearer to the proximal end of the optical in-vivo insertion probe than the sheath 20.

The sheath 20 is a tubular body closed at its front end and has a shaft lumen 67, extended from the proximal end of the sheath toward the front end thereof, for accommodating the data acquisition shaft. The sheath 20 has a sheath tube, a kink-resistant protector 25 disposed at a proximal end of the sheath tube, a base portion tube 23 fixed to a proximal portion of the protector 25, and a tube huh 24 fixed to a proximal end of the base portion tube 23.

In the in-vivo insertion probe 2 of this embodiment, the sheath tube is constructed of an inner tube 70, an intermediate tube 63, and an outer tube 65. The protector 25 is fixed to the proximal portion of the sheath tube. The base portion tube 23 extended to the proximal side of the optical in-vivo insertion probe by a predetermined length is fixed to the proximal portion of the protector 25. The tube hub 24 is fixed to the proximal portion of the base portion tube 23.

As shown in FIGS. 5 and 6, the data acquisition shaft 60 has the drive transmission hollow shaft 62, the optical fiber 61 penetrating through the hollow shaft 62 and having the chip portion 64 exposed from the front end portion of the hollow shaft 62, a connector connected to a proximal portion of the optical fiber 61, and a connection member 26 connecting a proximal portion of the hollow shaft 62 and the connector to each other. The data acquisition shaft 60 is rotated by the rotational force imparted thereto by the connector. The drive transmission hollow shaft 62 is a hollow body extended by a predetermined length and has an inner lumen portion penetrating therethrough from its proximal end to its front end. The inner lumen portion is capable of accommodating the optical fiber. As the drive transmission shaft 62, it is possible to use a coil, a round wire or a flat metal wound in a single layer or a multilayer in the form of a coil or a blade, and a resin tube coated with a metallic rigidity imparting body or embedded therein.

As the optical fiber 61, it is possible to use a known solid optical fiber which can be extended by a predetermined length. As the optical fiber, for example, a single mode optical fiber can be used. It is preferable to coat an outer surface of a clad of the optical fiber with a resin material called jacket. As shown in FIG. 6, a chip portion 64 is optically connected to the front end of the optical fiber 61. In the in-vivo insertion probe of this embodiment, a lens is used as the chip portion 64.

The method of using the in-vivo intravascular blood replacing liquid of the present invention is described below.

The in-vivo insertion probe 2 is used by connecting a proximal portion (connector portion of data acquisition shaft and proximal portion of operating holding member of operation member 21) thereof to an external device (not shown).

The external device is connected to the connector of the data acquisition shaft, and has a driving source for rotating the data acquisition shaft at a high speed, an optical source for supplying light to the optical fiber of the data acquisition shaft, and an image display function of forming an image by using light sent from the chip portion (lens portion) of the data acquisition shaft.

In using the in-vivo insertion probe, by using the guiding catheter where the in-vivo insertion probe 2 whose proximal portion has been connected to the external device is inserted, the in-vivo insertion probe is inserted into an intravascular portion to be diagnosed. As shown in FIG. 1, by using the prefilled syringe connected to the branch hub of the guiding catheter 3, the in-vivo intravascular blood replacing liquid is injected into a blood vessel. Thereby blood at the intravascular portion disposed forward by a predetermined interval from the guiding catheter is carried away by the in-vivo intravascular blood replacing liquid. As a result, the intravascular portion to be diagnosed is filled with the in-vivo intravascular blood replacing liquid. Thereafter the external device is driven to rotate the data acquisition shaft. Then in-vivo information is obtained from the chip portion rotating together with the shaft. In performing axial scan by means of the in-vivo insertion probe, the probe is moved axially at the intravascular portion to be diagnosed. Thereby new in-vivo information can be obtained.

Description is made below on another example of the in-vivo insertion probe for image diagnosis for which the in-vivo intravascular blood replacing liquid of the present invention is used.

An in-vivo insertion probe 100 of this embodiment is constructed by applying the in-vivo insertion probe of the present invention to an ultrasonic in-vivo insertion probe.

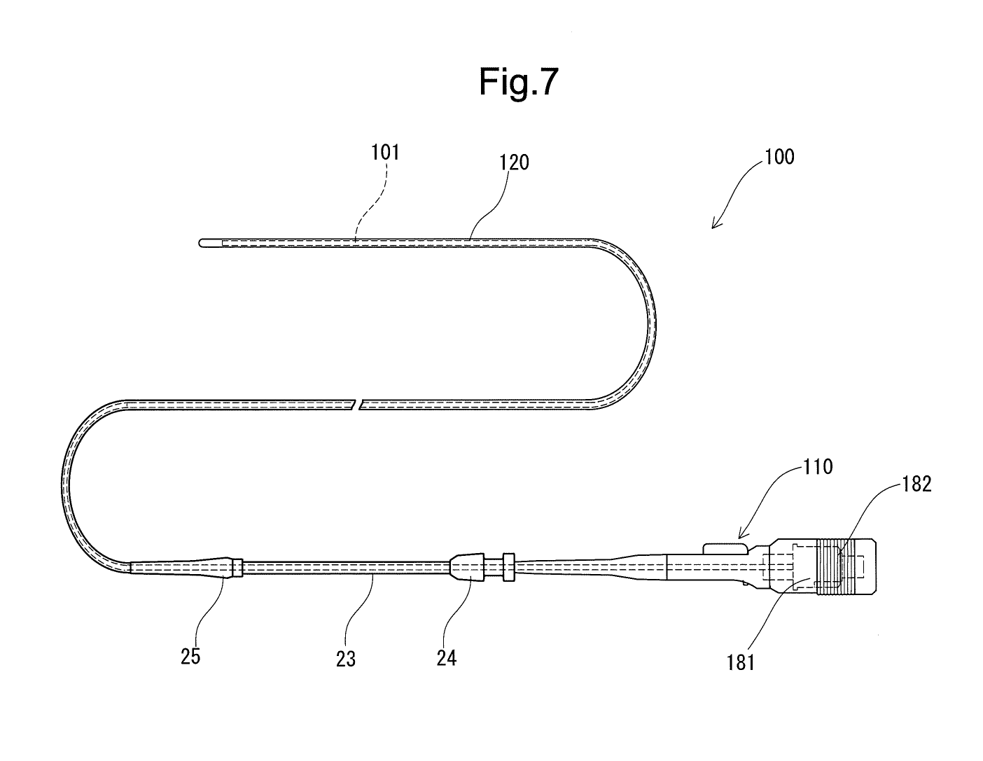

As shown in FIG. 7, the ultrasonic in-vivo insertion probe 100 of this embodiment comprises a sheath 120 to be inserted into a body cavity and a data acquisition shaft 101 inserted into the sheath 120. The sheath 120 is the same as that described above.

The data acquisition shaft 101 of this embodiment has a drive transmission hollow shaft 102, an ultrasonic vibrator 104 fixed to a front end portion of the hollow shaft 102, and a connector 110 connectable to a connection portion of an external device. The data acquisition shaft 101 is rotated by a rotational force imparted thereto by the connector 110.

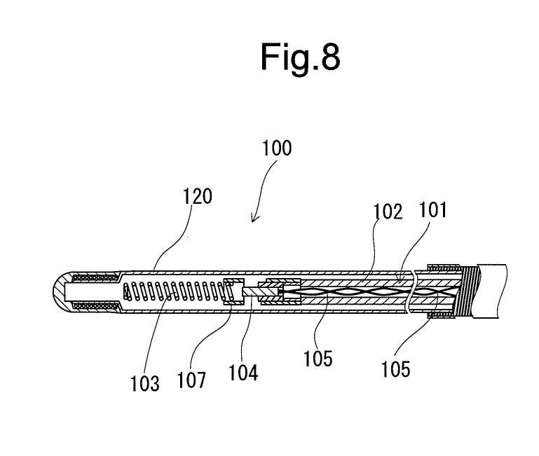

As shown in FIG. 8, as a chip portion, a transducer 104 having the function of an ultrasonic vibrator for sending and receiving ultrasonic waves is used for the data acquisition shaft 101. The data acquisition shaft 101 has a transducer housing 107 for accommodating the transducer 104 at its front end portion. The housing 107 is a tubular body having an open portion for exposing the transducer 104. The housing is fixed to the front end portion of the hollow shaft 102 at its proximal portion. A rotation stabilization member 103 extended toward the front end of the ultrasonic in-vivo insertion probe is mounted on a front end portion of the housing 107. As the rotation stabilization member, a coiled body as shown in FIG. 8 is preferable. The drive transmission hollow shaft 102 is a hollow body having a predetermined length and a lumen penetrating therethrough from its proximal end to its front end.

As shown in FIG. 8, the hollow shaft 102 incorporates a signal line 105 consisting of two twisted lead wires. A front end of the signal line 105 is connected to a vibrator of the transducer 104. A rear end of the signal line 105 is connected to a receptacle (not shown) of the connector 110. The connector 110 has a connector housing 181 and an annular elastic member 182 provided on an outer surface of the connector housing. The data acquisition shaft 101 of the in-vivo insertion probe 100 of this embodiment also rotates.

The external driving device (not shown) to which the in-vivo insertion probe 100 is connected has a function of picking up signals transmitted from a driving source including a motor and the probe. The external driving device is electrically connected to a console having a sending and receiving circuits and an image display device.

As with the above-described image diagnosis to be performed by using light, the in-vivo intravascular blood replacing liquid of the present invention is used for the image diagnosis to be performed by using ultrasonic waves.

### Examples

1. The following substances were prepared as additives.
1) Glycyrrhizin acid monoammonium (GLZA): produced by Maruzen Pharmaceuticals Co., Ltd.
2) Thiamine chloride hydrochloride: produced by DSM Japan K.K.
3) Gelatin: produced by Jellice Co., Ltd.
4) Polyvinylpyrrolidone: molecular weight 40,000, produced by Sigma-Aldrich Corporation
5) Polyethylene glycol 400: molecular weight 400 (PEG400: brand name), produced by Kanto Chemical Co., Inc.

2. The following apparatuses were prepared:
1) OFDI intravascular image diagnosis apparatus: LUNAWAVE (registered trademark) produced by Terumo Corporation
2) Guiding catheter (6Fr, inner diameter: 1.8 mm, outer diameter: about 2.0 mm): Heartrail (registered trademark) produced by Terumo Corporation
3) Guiding catheter (5Fr, inner diameter: 1.5 mm, outer diameter: about 1.7 mm): Heartrail (registered trademark) produced by Terumo Corporation
4) Guide wire: Runthrough ((registered trademark) produced by TERUMO CORPORATION
5) In-vivo insertion probe for image diagnosis: FirstView (registered trademark) produced by Terumo Corporation

3. A lactic acid buffer solution was prepared as follows:

6.0 g of sodium chloride, 0.3 g of potassium chloride, 0.2 g of calcium chloride dehydrate, and 6.2 g of an L-sodium lactate solution were weighed. Water was added to the mixture of the above-described substances to dissolve them therein. Thereafter water was added to the solution to set the volume thereof to exactly 1 L.

4. Thiamine chloride hydrochloride was formed as follows:

1 g of thiamine chloride hydrochloride was dissolved in water to set the volume of the solution to 100 mL. In this manner, the thiamine chloride hydrochloride solution was formed.

### Examples and Comparison Examples

An in-vivo intravascular blood replacing liquid (flush solution) of each of the examples and comparison examples was formed or prepared as follows.

### Example 1

50 ml of water was added to 25 g of the monoammonium glycyrrhizinate to prepare a monoammonium glycyrrhizinate solution having a concentration of 0.5 mg/mL. 1 g of glucose and 0.225 g of sodium chloride were added to the monoammonium glycyrrhizinate solution to form an aqueous monoammonium glycyrrhizinate solution. The thiamine chloride hydrochloride solution was added to 10 ml of the prepared aqueous monoammonium glycyrrhizinate solution to form the in-vivo intravascular blood replacing liquid (flush solution). The mol ratio between the monoammonium glycyrrhizinate and the thiamine chloride hydrochloride was set to 1:1. The viscosity of the obtained flush solution at 25 degrees C. was 1.4 mPa·s.

### Example 2

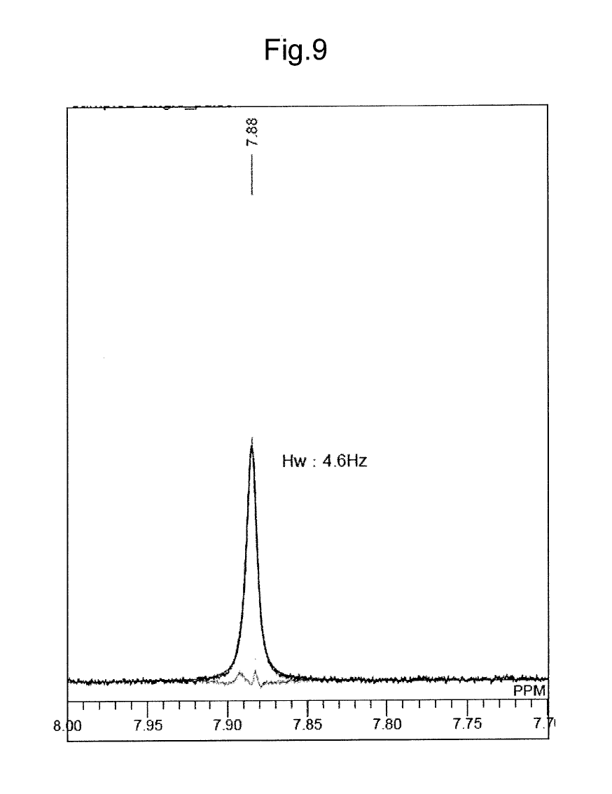

The thiamine chloride hydrochloride solution was added to the aqueous monoammonium glycyrrhizinate solution formed in the example 1 to form the in-vivo intravascular blood replacing liquid (flush solution). The mol ratio between the monoammonium glycyrrhizinate and the thiamine chloride hydrochloride was set to 1:2. The viscosity of the obtained flush solution at 25 degrees C. was 1.7 mPa·s. The ¹H-NMR spectrum of the flush solution of the example 2 was measured. The result of the measurement is as shown in FIG. 9. The spectrum shown with the black line (not grey line) in FIG. 9 is the ¹H-NMR spectrum derived from thiamine. The half width of the peak derived from the thiamine of the flush solution is as shown in table 2.

### Example 3

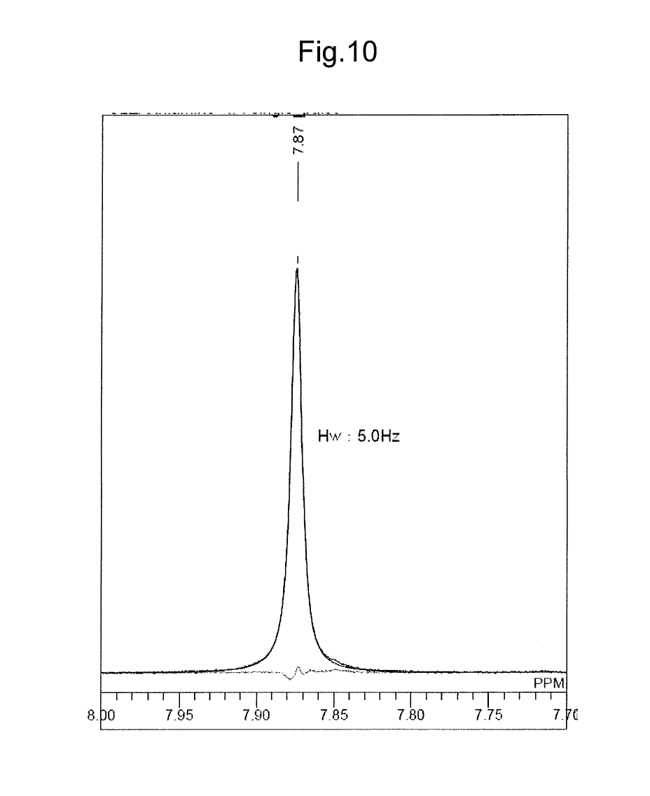

The thiamine chloride hydrochloride solution was added to the aqueous monoammonium glycyrrhizinate solution formed in the example 1 to form the in-vivo intravascular blood replacing liquid (flush solution). The mol ratio between the aqueous monoammonium glycyrrhizinate and the thiamine chloride hydrochloride was set to 1:4. The viscosity of the obtained flush solution at 25 degrees C. was 2.0 mPa·s. The ¹H-NMR spectrum of the flush solution of the example 3 was measured. The result of the measurement is as shown in FIG. 10. The spectrum shown with the black line (not grey line) in FIG. 10 is the ¹H-NMR spectrum derived from the thiamine. The half width of the peak derived from the thiamine of the flush solution is as shown in table 2.

### Example 4

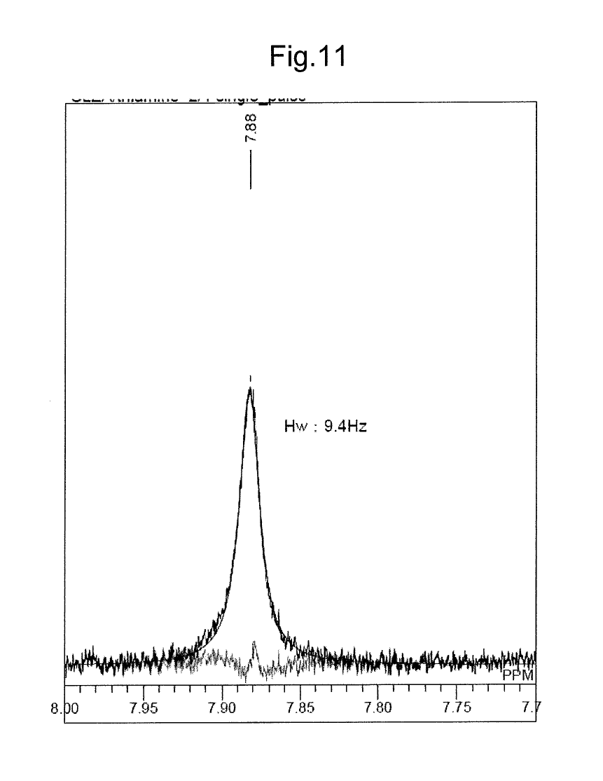

The thiamine chloride hydrochloride solution was added to the aqueous monoammonium glycyrrhizinate solution formed in the example 1 to form the in-vivo intravascular blood replacing liquid (flush solution). The mol ratio between the monoammonium glycyrrhizinate and the thiamine chloride hydrochloride was set to 2:1. The viscosity of the obtained flush solution at 25 degrees C. was 2.3 mPa·s. The ¹H-NMR spectrum of the flush solution of the example 4 was measured. The result of the measurement is as shown in FIG. 11. The spectrum shown with the black line (not grey line) in FIG. 11 is the ¹H-NMR spectrum derived from the thiamine. The half width of the peak derived from the thiamine of the flush solution is as shown in table 2.

### Example 5

By adding 50 mg of the gelatin to 50 mL of the flush solution of the example 1 and dissolving the gelatin in the flush solution, the in-vivo intravascular blood replacing liquid (flush solution) was formed. The viscosity of the flush solution at 25 degrees C. was 1.0 mPa·s.

### Example 6

By adding 100 mg of the gelatin to 50 mL of the flush solution of the example 1 and dissolving the gelatin in the flush solution, the in-vivo intravascular blood replacing liquid (flush solution) was formed. The viscosity of the flush solution at 25 degrees C. was 1.1 mPa·s.

### Example 7

By adding 200 mg of the gelatin to 50 mL of the flush solution of the example 1 and dissolving the gelatin in the flush solution, the in-vivo intravascular blood replacing liquid (flush solution) was formed. The viscosity of the flush solution at 25 degrees C. was 1.1 mPa·s.

### Example 8

By adding 1 g of the PEG400 to 50 mL of the flush solution of the example 4 and dissolving the PEG400 in the flush solution, the in-vivo intravascular blood replacing liquid (flush solution) was formed. The viscosity of the flush solution at 25 degrees C. was 1.5 mPa·s.

### Example 9

By adding 3 g of PEG400 to 50 mL of the flush solution of the example 4 and dissolving PEG400 in the flush solution, the in-vivo intravascular blood replacing liquid (flush solution) was formed. The viscosity of the flush solution at 25 degrees C. was 1.5 mPa·s.

### Example 10

By adding 6 g of PEG400 to 50 mL of the flush solution of the example 4 and dissolving the PEG400 in the flush solution, the in-vivo intravascular blood replacing liquid (flush solution) was formed. The viscosity of the flush solution at 25 degrees C. was 1.7 mPa·s.

### Comparison Example 1

As a comparison example 1, saline was used. The viscosity of the flush solution at 25 degrees C. was 1.0 mPa·s.

### Comparison Example 2

As a comparison example 2, a lactate Ringer solution was used. The viscosity of the flush solution at 25 degrees C. was 1.0 mPa·s.

### Comparison Example 3

The lactic acid buffer solution was added to the weighed amount of the glycyrrhizin acid monoammonium to prepare a solution having a concentration of 0.20 mg/mL. In this manner, the flush solution was obtained. The viscosity of the obtained flush solution at 25 degrees C. was 1.0 mPa·s.

### Comparison Example 4

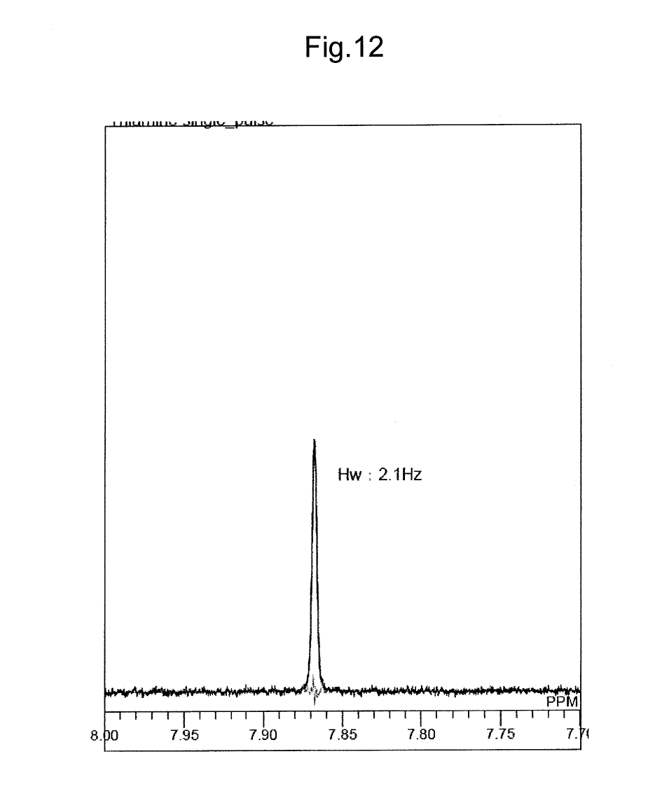

The lactic acid buffer solution was added to the weighed amount of the thiamine chloride hydrochloride to prepare a solution having a concentration of 0.20 mg/mL. In this manner, the flush solution was obtained. The viscosity of the obtained flush solution at 25 degrees C. was 0.8 mPa·s. The ¹H-NMR spectrum of the flush solution of the comparison example 4 was measured. The result of the measurement is as shown in FIG. 12. The spectrum shown with the black line (not grey line) in FIG. 12 is the ¹H-NMR spectrum derived from the thiamine. The half width of the peak derived from the thiamine is as shown in table 2.

### Example 11

The thiamine chloride hydrochloride solution was added to the aqueous monoammonium glycyrrhizinate solution formed in the example 1 to form the in-vivo intravascular blood replacing liquid (flush solution). The mol ratio between the monoammonium glycyrrhizinate and the thiamine chloride hydrochloride was set to 8:1. The viscosity of the obtained flush solution at 25 degrees C. was 3.2 mPa·s.

### Example 12

The thiamine chloride hydrochloride solution was added to the aqueous monoammonium glycyrrhizinate solution formed in the example 1 to form the in-vivo intravascular blood replacing liquid (flush solution). The mol ratio between the monoammonium glycyrrhizinate and the thiamine chloride hydrochloride was set to 1:6. The viscosity of the obtained flush solution at 25 degrees C. was 3.0 mPa·s.

### Comparison Example 5

Low molecular weight dextran (product name: “Otsuka Dextran L injection” produced by Otsuka Pharmaceutical Factory Co., Ltd.) having an average molecular weight of 40,000 was used to form the in-vivo intravascular blood replacing liquid (flush solution). The viscosity of the flush solution at 25 degrees C. was 4.8 mPa·s.

### Comparison Example 6

Omnipaque (registered trademark) 300 injection syringe (non-ionic contrast agent produced by Daiichi Sankyo Company, Ltd.) was prepared to form the in-vivo intravascular blood replacing liquid (the flush solution). The viscosity of the flush solution at 20 degrees C. was 13.3 mPa·s.

The viscosities of the above-described flush solutions were measured by using a viscometer.

### Experiment 1

### Evaluation of In-Vitro Image Diagnosis

By using the in-vivo intravascular blood replacing liquids (flush solutions) of the examples and comparison examples, evaluations of image diagnoses were conducted by using a model blood vessel. As the model blood vessel, the blood of a pig was applied to a silicone tube by using a constant flow rate pump. When a state in which the inner diameter of the silicone tube could be seen for not less than three seconds continued, image diagnoses were judged as good. Each flush solution was fed to each of the outer diameter-different guiding catheters in a state where the in-vivo insertion probe for image diagnosis and the guide wire were inserted into each guiding catheter. A syringe pump was used to feed the flush solutions.

The experimental conditions of the experiment 1 were as follows:

Flush solution feeding conditions:

Pig blood feeding speed: 250 mL/minute

Flush solution feeding speed: 150 mL/minute

Feeding amount of flush solution: 20 mL

The results of the in vitro experiment made on the image diagnostic performance were as shown in table 1.

TABLE 1

| Flush | Viscosity | Image diagnostic | Image diagnostic |
|---|---|---|---|
| solution | (mPa · s) | performance: 6Fr | performance: 5Fr |
| Example 1 | 1.4 | Good | Good |
| Example 2 | 1.7 | Good | Good |
| Example 3 | 2.0 | Good | Good |
| Example 4 | 2.3 | Good | Good |
| Example 5 | 1.0 | Good | Good |
| Example 6 | 1.1 | Good | Good |
| Example 7 | 1.1 | Good | Good |
| Example 8 | 1.5 | Good | Good |
| Example 9 | 1.5 | Good | Good |
| Example 10 | 1.7 | Good | Good |
| Example 11 | 3.2 | Good | Bad |
| Example 12 | 3.0 | Good | Bad |
| Comparison | 1.0 | Bad | Bad |
| example 1 | | | |
| Comparison | 1.0 | Bad | Bad |
| example 2 | | | |
| Comparison | 1.0 | Bad | Bad |
| example 3 | | | |
| Comparison | 0.8 | Bad | Bad |
| example 4 | | | |
| Comparison | 4.8 | Bad | Bad |
| example 5 | | | |
| Comparison | 13.3 | Good | Bad |
| example 6 | | | |

As shown in table 1, the flush solutions of all of the examples 1 through 12 in which the 6Fr guiding catheter was used allowed images to be seen clearly for not less than five seconds after the flush solutions were applied to the in-vivo insertion probe and the guiding catheters. Thus the flush solutions of the present invention proved to be effective. The flush solutions of the examples 1 through 10 in which the 5Fr guiding catheter was used allowed images to be seen clearly for not less than five seconds after the flush solutions were applied to the in-vivo insertion probe and the guiding catheters.

On the other hand, although the flush solutions of the comparison examples 1 through 5 had the viscosities close to those of the flush solutions of the examples, the image diagnostic performance thereof was evaluated as bad. Therefore it is conceivable that the flush solutions in the present invention achieved good image diagnostic performance owing to a factor different from the viscosity.

The flush solutions of the comparison examples 5, 6 are commonly used now in angiography to be performed by using the OFDI intravascular image diagnosis apparatus. The image diagnostic performance of the flush solution of the comparison example 6 was evaluated. As a result, in the case in which the 6Fr guiding catheter was used, good image diagnostic performance was shown, whereas in the case in which the 5Fr guiding catheter was used, good image diagnostic performance was not obtained. Conceivably, because the viscosity of the flush solution was high, an intended speed could not be achieved or an intended amount of the flush solution could not be injected into the blood vessel. The flush solution of the comparison example 5 did not allow good image diagnostic performance to be obtained in both the cases where the 6Fr and 5Fr guiding catheters were used. On the other hand, it was confirmed that the flush solutions of the present invention having low viscosities allowed good image diagnostic performance to be obtained even in the use of the 5Fr guiding catheter.

### Experiment 2

An experiment of the viscosity of the flush solution and the injection resistance value were conducted.

As the flush agent of the experiment 2, those of the examples 1 through 6 and those of the comparison example 1, 3, 4, and 6 were used. After the flush agents were filled in syringes having a volume of 20 mL, the syringes were connected to 5Fr and 6Fr guiding catheters. In a state where the in-vivo insertion probe for image diagnosis and the guide wire were inserted into the guiding catheters, the flush agents were pressed out of the syringes by using AUTOGRAPH (produced by Shimazu Corporation). In the measurement of the injection resistance value, a maximum value of forces applied to the plunger of each syringe at the time when each flush agent was pressed out of each syringe was set as the injection resistance value in the examples and the comparison examples. The press-out speed of each flush agent was set to 100 mL/minute. The results were as shown in table 2.

The ¹H-NMR spectrum of the flush solutions of the examples and those of the comparison examples were measured. The half widths of the peaks derived from the thiamine of the flush solutions were as shown in table 2.

The conditions in which the ¹H-NMR spectra were measured were as follows:

Measuring method: single pulse method, relaxation time measurement
Measured nuclear frequency: 600.1753 MHz (¹H nucleus)
Spectrum width: 12.0 kHz
Pulse width: 5.0 μsec (45 degree-pulse)
Repetition period of time:

ACQTM: 8.724 sec. PD: 20.0 sec. (single pulse)

ACQTM: 1.365 sec. PD: 10.0 sec. (relaxation time measurement)

Observation point:

104858 data point: 104858 (single pulse)

16384 data point: 16384 (relaxation time measurement)

Solvent: deuterium oxide
Reference material: water in deuterium oxide (internal reference: 4.65 ppm)
Temperature: room temperature
Number of rotations of specimens: 15.0 Hz
¹H-NMR measuring apparatus:

ECA600, JEOL produced by RESONANCE Co., Ltd.

TABLE 2

| | Half width of | | | |
|---|---|---|---|---|
| | peak (7.9 ppm) | | Injection | Injection |
| | derived from | | resistance | resistance |
| Flush | thiamine in NMR | Viscosity | 6Fr | 5Fr |
| solution | measurement | mPa · s | (N) | (N) |
| Example 1 | 4.0 | 1.4 | 15.4 | 47.7 |
| Example 2 | 4.6 | 1.7 | 16.1 | 49.6 |
| Example 3 | 5.0 | 2.0 | 18.3 | 52.4 |
| Example 4 | 9.4 | 2.3 | 19.9 | 56.5 |
| Example 5 | 20.5 | 1.2 | 16.5 | 41.1 |
| Example 6 | 16.2 | 3.0 | 30.2 | 76.7 |
| Comparison | — | 1.0 | 9.3 | 38.3 |
| example 1 | | | | |
| Comparison | — | 1.0 | 9.7 | 39.9 |
| example 3 | | | | |
| Comparison | 2.1 | 0.8 | 9.6 | 39.0 |
| example 4 | | | | |
| Comparison | — | 13.3 | 50.0 | 224.9 |
| example 6 | | | | |

As shown in table 2, it was found that there was an increase in the injection resistance value in dependence on the viscosity when the 6Fr and 5Fr guiding catheters were used. It was also found that the injection resistance value at the time when the 5Fr guiding catheter was used was not less than four times as high as the injection resistance value at the time when the 6Fr guiding catheter was used. The injection resistance values in the comparison examples 6 were very high when the 5Fr guiding catheter was used. These results indicate that when the injection resistance value becomes excessively high, there is a possibility that the flush agent cannot be injected at a constant speed. As a result, in the experiment 1, it was judged that the reason good image diagnostic performance was not obtained when the 5Fr guiding catheter was used for the flush solutions having high viscosities is caused by the high viscosity of the flush solution.

### Experiment 3

The image diagnostic performance of the flush solutions of the example 1 and the comparison examples 5 and 6 were checked by using an animal. After the 6Fr and 5Fr guiding catheters were inserted respectively into a blood vessel of a pig, a guide wire (outer diameter: 0.35 mm) and FDI catheter were inserted into the catheter. Each flush solution was injected into the blood vessel through each catheter. Of the obtained intravascular images, those allowing the inner wall of the blood vessel to be seen with eyes were defined as CLEAR FRAME. CLEAR FRAME time periods were calculated from the obtained intravascular images.

The results are as shown in table 3.

TABLE 3

| | |
|---|---|
| Flush | CLEAR FRAME TIME (sec) |

| solution | Catheter 6Fr | Catheter 5Fr |
|---|---|---|
| Example 1 | 3.7 | 3.5 |
| Comparison | 2.45 | 0.35 |
| example 5 | | |
| Comparison | 3.6 | — |
| example 6 | | |

The results were that the CLEAR FRAME time period obtained in the example 1 of the present invention was longer than that obtained in the comparison example 5 and was equal to or longer than that obtained in the comparison example 6. The CLEAR FRAME time period obtained when the 5Fr guiding catheter was used was equal to that obtained when the 6Fr guiding catheter was used. The results of the experiment 3 indicate that although the flush solution of the example 1 had a low viscosity, it allowed image diagnostic performance to be obtained equivalently to a flush solution having a high viscosity. The results of the experiment 3 also indicate that although the guiding catheter having a small inner diameter is used, the flush solution of the example 1 allowed good image diagnostic performance to be obtained.

1. An in-vivo intravascular blood replacing liquid injected into a blood vessel to replace blood at an in-vivo intravascular portion to be inspected therewith in making an in-vivo intravascular inspection,
wherein said blood replacing liquid comprises an aqueous medium unharmful for a living body and a cationic compound and an anionic compound, added thereto, which are unharmful for said living body; and
said blood replacing liquid includes an ion complex formed of said cationic compound and said anionic compound.

2. An in-vivo intravascular blood replacing liquid according to claim 1, wherein a half width of a peak derived from said cationic compound in a ¹H-NMR spectrum is 3 to 15 Hz.

3. An in-vivo intravascular blood replacing liquid according to claim 1, wherein said half width of said peak derived from said cationic compound in a ¹H-NMR spectrum in said in-vivo intravascular blood replacing liquid is 1.5 to 20 times as large as a half width of a peak derived from said cationic compound alone in said ¹H-NMR spectrum.

4. An in-vivo intravascular blood replacing liquid according to claim 1, wherein said anionic compound is at least one selected from a group consisting of glycyrrhizin acids, hyaluronic acids, chondroitin sulfates, alginic acids, ammonium sulfates, dextran sulfates, and glucuronic acids.

5. An in-vivo intravascular blood replacing liquid according to claim 1, wherein said cationic compound is at least one selected from a group consisting of basic amino acids or derivatives thereof, chitin or chitosan or derivatives thereof, and basic compounds.

6. An in-vivo intravascular blood replacing liquid according to claim 1, wherein combinations of said cationic and anionic compounds are mixtures of glycyrrhizin acids and basic compounds or derivatives thereof, mixtures of chitin or chitosan or derivatives thereof and hyaluronic acid or derivatives thereof, and mixtures of chondroitin sulfate or derivatives thereof and said basic compounds.

7. An in-vivo intravascular blood replacing liquid according to claim 1, wherein said cationic compound is thiamines.

8. An in-vivo intravascular blood replacing liquid according to claim 1, wherein a combination of said cationic and anionic compounds is a mixture of glycyrrhizin acids and thiamines.

9. An in-vivo intravascular blood replacing liquid according to claim 1, wherein said aqueous medium is sterile water, saline or a buffer solution.

10. An in-vivo intravascular blood replacing liquid according to claim 1, which has a viscosity of not more than 2 mPa·s at not more than 30 degrees C.

11. An in-vivo intravascular blood replacing liquid formulation comprising a medical container and an in-vivo intravascular blood replacing liquid filled inside said medical container,
wherein said blood replacing liquid comprises an aqueous medium unharmful for a living body and a cationic compound and an anionic compound, added thereto, which are unharmful for said living body; and
said blood replacing liquid includes an ion complex formed of said cationic compound and said anionic compound.

12. A prefilled syringe comprising an outer cylinder, a gasket accommodated inside said outer cylinder, a sealing part for sealing a front end portion of said outer cylinder, and an in-vivo intravascular blood replacing liquid filled inside said outer cylinder,
wherein said blood replacing liquid comprises an aqueous medium unharmful for a living body and a cationic compound and an anionic compound, added thereto, which are unharmful for said living body; and
said blood replacing liquid includes an ion complex formed of said cationic compound and said anionic compound.

13. A prefilled syringe according to claim 12, which is subjected to heat sterilization with said intravascular blood replacing liquid being filled therein.
